(19) United States
(12) Patent Application Publication
(10) Pub. No.: US 2024/0399084 A1
(43) Pub. Date: Dec. 5, 2024

(54) OXYGEN SENSOR COMPENSATION FOR FRACTIONAL OXYGEN DRIFT IN MECHANICALLY VENTILATED PATIENTS

(71) Applicant: KONINKLIJKE PHILIPS N.V., Eindhoven (NL)

(72) Inventors: Isha Malekar, Cambridge, MA (US); Francesco Vicario, Cambridge, MA (US); Roberto Buizza, Eindhoven (NL)

(21) Appl. No.: 18/591,103

(22) Filed: Feb. 29, 2024

Related U.S. Application Data
(60) Provisional application No. 63/469,584, filed on May 30, 2023.

(51) Int. Cl. A61M 16/00

(52) U.S. Cl. CPC A61M 16/0075; A61M 16/0003; A61M 16/024; A61M 2205/3327; A61M 2205/3379; A61M 2205/50; A61M 2230/43

(57) ABSTRACT

An embodiment includes a ventilator system that corrects oxygen sensor measurements, for example derived from an oxygen sensor in a mixing chamber of an indirect calorimeter or oxygen consumption (VO₂) monitoring device. In an example, an embodiment determines fractional inspired oxygen drift as supplied by a ventilator and utilizes the change or change rate in fractional inspired oxygen over time to correct VO₂ determinations made by the system.

### CROSS-REFERENCE TO RELATED APPLICATIONS

This patent application claims the priority benefit under 35 U.S.C. § 119(e) of U.S. Provisional Application No. 63/469,584, filed on May 30, 2023, the contents of which are herein incorporated by reference.

### BACKGROUND

### 1. Field

The disclosed subject matter generally pertains to correcting oxygen sensor measurements used for determining oxygen consumption (VO₂) with greater accuracy. Certain disclosed subject matter relates to use of a correction to measured oxygen, such as fractional inspired oxygen (FiO₂), sensed within a mixing chamber of an indirect calorimetry system such as included in a patient ventilation circuit.

### 2. Description of the Related Art

Oxygen consumption (VO₂) is a clinically important measurement in acute patient management, such as in mechanically ventilated patients requiring fractional inspired oxygen (FiO₂) higher than ambient (i.e., FiO₂ greater than 21%). Oxygen consumption has several clinical applications such as in infection detection, nutrition management, as well as hemodynamic and ventilation management. Furthermore, oxygen consumption measurements are of particular interest in critical patients since oxygen consumption is intimately related to the transport of oxygen from the lungs to the tissues that need it. As such, oxygen consumption monitoring provides significant insights into the cardiovascular and metabolic status of a patient.

Oxygen consumption is defined as the difference between the inhaled and the exhaled volume of oxygen. Both the inhaled and exhaled oxygen volumes can be computed (by direct measurement or indirect determination) to infer oxygen consumption. Conventionally, oxygen consumption monitoring devices contain a mixing chamber to measure gas concentrations, where inspired and expired gases are sampled. Contained in such mixing chambers are gas measurement sensors (oxygen, nitrogen, and/or carbon dioxide sensors), flow sensors, and sensors for compensation of pressure, temperature, and relative humidity.

Techniques such as the Haldane transformation (normalizing based on nitrogen) can compute oxygen consumption using the measurements of fractional inspired oxygen (FiO₂), fractional inspired carbon dioxide (FiCO₂), fractional expired oxygen (FeO₂), and fractional expired carbon dioxide (FeCO₂), logged by mixing chamber sensors. In some systems, oxygen consumption is computed using inhaled and exhaled gas concentrations sampled at different times.

### SUMMARY

In supplemental oxygen systems, such as a patient ventilator used for invasive patient ventilation, oxygen consumption is computed using inhaled and exhaled gas concentrations sampled at different times. Use of oxygen consumption calculations such as the Haldane transformation become inaccurate in such systems because this approach assumes that the gas concentration measurements are simultaneous, accounting for any variation in supplied oxygen, which is not the case for inhalation and exhalation measurements. The time discontinuity of gas concentration measurements leads to errors in determined oxygen consumption, particularly with supplemental oxygenation, measured as fractional inspired oxygen (FiO₂) that is variably supplied by a ventilator.

Accordingly, an embodiment overcomes the problem of inhalation and exhalation gas concentration measurements separated in time, particularly in the context of variability in supplemental oxygen (“FiO₂ drift”). By accounting for these factors, an embodiment eliminates the dependency of oxygen consumption measurement accuracy on stability of FiO2 values.

In summary, an embodiment provides a ventilator system, comprising: a ventilator comprising a first sensor; one or more mixing chambers; one or more other sensors disposed in the one or more mixing chambers; a set of one or more processors; and a non-transitory storage medium storing code executable by the set of one or more processors. In an embodiment, the code comprises code that obtains a first fractional inspired oxygen concentration (FiO₂) value of breathable gas provided by the ventilator to a patient, measured by the first sensor at a first time and a second FiO₂ value of breathable gas provided by the ventilator to the patient, measured by the first sensor at a second time. The code includes code that obtains a third FiO₂ value of breathable gas provided by the ventilator to the patient, measured by the one or more other sensors at the first time, and code that obtains a fractional expired oxygen concentration (FeO₂) value of breathable gas exhaled by the patient, measured by the one or more other sensors at the second time.

In an embodiment, the code includes code that determines a change in FiO₂ between the first FiO₂ value and the second FiO₂ value, determines a correction to one or more of the third FiO₂ value and the FeO₂ value based on the determined change in FiO₂; and adjusts one or more of the third FiO₂value and the FeO₂ value based on the correction.

In an embodiment, the system comprises code that determines a difference between: (a) the third FiO₂ value and an adjusted FeO₂ value; (b) an adjusted third FiO₂ value and the FeO₂ value; or (c) the adjusted third FiO₂ value and the adjusted FeO₂ value. In an embodiment, the code comprises code that determines an oxygen consumption of the patient based on the difference.

In an embodiment, the first time occurs during a first time period comprising inspiration by the patient and the second time occurs during a second time period comprising expiration by the patient. In an embodiment, the first time comprises a first time sampling window, wherein the first and third FiO₂ values are average values over the first time sampling window. In an embodiment, the second time comprises a second time sampling window, wherein the second FiO₂ value and the FeO₂ value are average values over the second time sampling window.

In an embodiment, the code that adjusts comprises code that outputs a correction value to increase or decrease the third FiO₂ value by a magnitude that is equivalent to the difference between the first FiO₂ value measured at the first time and the second FiO₂ value measured at the second time.

In an embodiment, the code that determines a correction comprises code that identifies a real-time change in the first FiO₂ value provided by the first sensor over time. In an embodiment, the real-time change value is derived from a time series of oxygen measurements by the first sensor comprising oxygen measurements made in the first and second times.

In an embodiment, the first and second time sampling windows each comprise a plurality of breathing cycles by the patient, and the first and second times are separated by a plurality of breathing cycles.

Another embodiment provides a computer implemented method of using one or more processors that execute instructions to perform actions, comprising: obtaining a first fractional inspired oxygen concentration (FiO₂) value of breathable gas provided by a ventilator to a patient, measured by a first sensor at a first time; obtaining a second FiO₂ value of breathable gas provided by the ventilator to the patient, measured by the first sensor at a second time; obtaining a third fractional FiO₂ value of breathable gas provided by the ventilator to the patient, measured by one or more other sensors at the first time; obtaining a fractional expired oxygen concentration (FeO₂) value of breathable gas exhaled by the patient, measured by the one or more other sensors at the second time; determining a change in FiO₂ between the first FiO₂ value and the second FiO₂ value; determining, using the set of one or more processors, a correction to one or more of the third FiO₂ value and the FeO₂ value based on the determined change in FiO₂; and adjusting one or more of the third FiO₂ value and the FeO₂ value based on the correction.

In an embodiment, the method includes performing actions of the ventilation system or component thereof, as described herein.

A further embodiment provides a computer program product, comprising: a non-transitory storage medium storing code executable by a set of one or more processors, the code comprising code included in the ventilator system or component operatively coupled thereto, as described herein.

As will become apparent from reviewing this specification, methods, devices, systems, and products are provided for implementing the various embodiments.

The foregoing is a summary and thus may contain simplifications, generalizations, and omissions of detail; consequently, those skilled in the art will appreciate that the summary is illustrative only and is not intended to be in any way limiting.

These and other features and characteristics of the example embodiments, as well as the methods of operation and functions of the related elements of structure and the combination thereof, will become more apparent upon consideration of the following description and the appended claims with reference to the accompanying drawings, all of which form a part of this specification. It is to be expressly understood, however, that the drawings are for the purpose of illustration and description only and are not intended as a definition of the limits of the invention.

### BRIEF DESCRIPTION OF THE DRAWINGS

FIG. 1 illustrates an example method according to an embodiment.

FIG. 2 illustrates an example system according to an embodiment.

FIG. 3 illustrates an example oxygen measurement correction according to an embodiment.

FIG. 4 illustrates a diagram of example system components according to an embodiment.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, e.g., through one or more intermediate parts or components, so long as a link occurs. As used herein, “operatively coupled” means that two or more elements are coupled so as to operate together or are in communication, unidirectional or bidirectional, with one another. As used herein, the term “number” shall mean one or an integer greater than one (i.e., a plurality). As used herein a “set” shall mean one or more.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well known structures, materials, or operations are not shown or described in detail to avoid obfuscation.

As opposed to known techniques for determining oxygen consumption (VO₂) relying on the assumption that fractional inspired oxygen (FiO₂) is measured at the same time as fractional expired oxygen (FeO₂) (i.e., the FiO₂ is unchanging or measured simultaneously with FeO₂), an embodiment determines the change in FiO₂ delivered and compensates oxygen gas concentrations measured to reduce the errors in VO₂ computation. The positive effect on VO₂ determinations from the disclosed embodiments is shown in an example from a VO₂ monitoring device designed for mechanically ventilated patients. The example VO₂ monitoring device contains one mixing chamber, which is intermittently swapped between inhalation limb and exhalation limb of a respiratory circuit. The example monitoring device computes VO₂ using the Haldane transformation. An embodiment may therefore be advantageously used to eliminate the errors posed by varying FiO₂.

The description now turns to the figures. The illustrated example embodiments will be best understood by reference to the figures. The following description is intended only by way of example, and simply illustrates certain example embodiments.

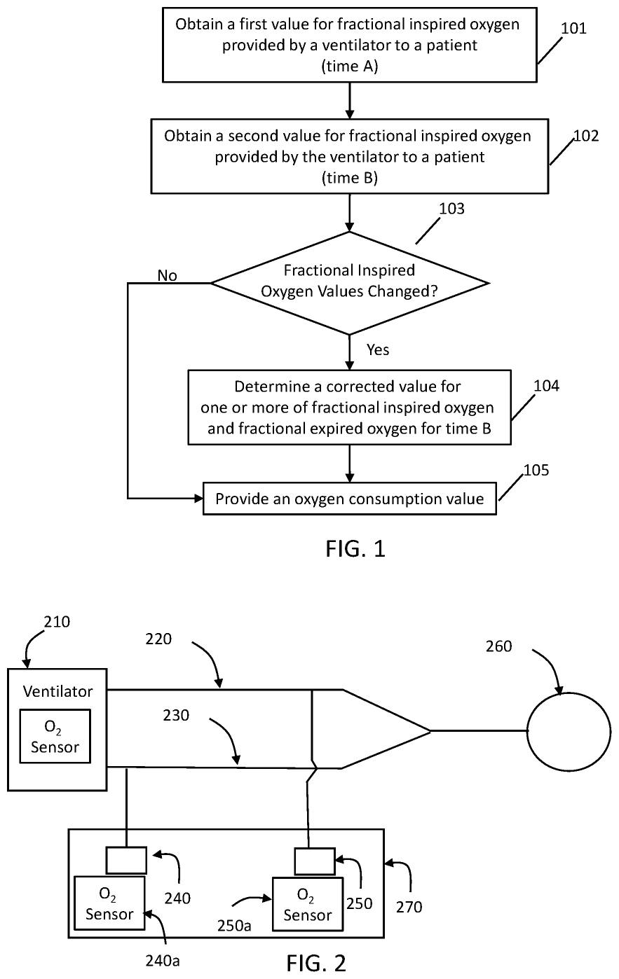

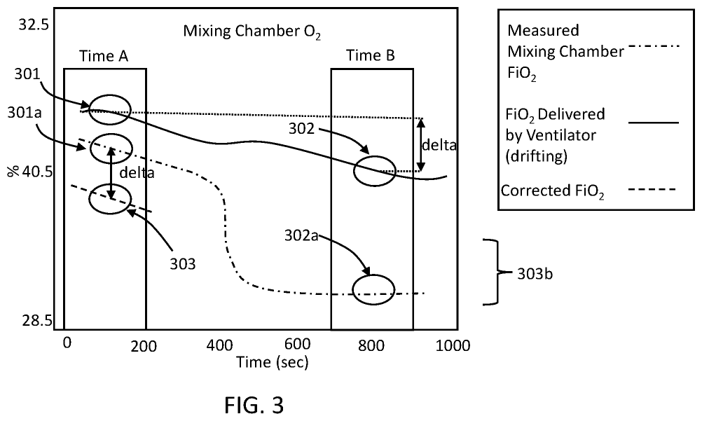

Referring to FIG. 1, an embodiment provides for determining a change in FiO₂ over time, e.g., time periods A and B as described in connection with FIG. 3. A benefit of such determination is to provide the determined change in FiO₂ needed to compensate inspired oxygen gas concentration measured by a mixing chamber.

In an embodiment, a change in FiO₂ is determined for example by obtaining ground truth measurements from a ventilator's oxygen sensor. In the example of FIG. 1, a first value for FiO₂ is obtained from a first sensor as indicated at 101, such as the oxygen sensor of a ventilator, at a first time, for example during inspiration. It is noted that the first value for FiO₂ may be an instantaneous or point value or an aggregate or averaged value over a time period or time window. Similarly at 102 a second value is obtained for FiO₂ from the sensor at a second time, for example the oxygen sensor of the ventilator during expiration. As with the first value, the second value may be a single or point in time value or an averaged value. It is noted here that while the ventilator's oxygen sensor is used by way of example, the first sensor may take other forms, for example use of an independent oxygen sensor associated with a component of the ventilation system, such as an inspiration limb of the patient circuit, etc.

At 103 it is determined whether a change, or drift, is detected in FiO₂ measurements between the first and second time. In a case where FiO₂ has changed, an embodiment may determine a correction to the FiO₂ for use in the providing of an oxygen consumption value, indicated at 104 and 105, respectively. Where FiO₂ has not changed (no drift is present), an embodiment may proceed to determine oxygen consumption without a correction.

FIG. 2 shows an example ventilator system according to an embodiment. Here, a mechanical ventilator 210 is shown operatively coupled to a patient 260 using a patient circuit comprised of inhalation 220 and exhalation 230 limbs. An inhalation side mixing chamber 250 and associated oxygen sensor 250a and exhalation side mixing chamber 240 and associated oxygen sensor 240a are illustrated with respective sampling lines. It is noted that a single mixing chamber switching its sampling between inspiration and expiration locations may be utilized.

Supplemental FiO₂ is delivered to patient 260 by mechanical ventilator 210. The one or more mixing chambers 240, 250 of a VO₂ monitoring device 270 used with ventilator 210 is measuring inhaled oxygen concentration (e.g., FiO₂ in the mixing chamber 250) during time interval A (for inspiration) and exhaled oxygen concentration (e.g., FeO₂ in mixing chamber 240) during time interval B (for exhalation).

Referring to FIG. 3, an example of FiO₂ drift correction is illustrated graphically. It can be noted that two measurements of FiO₂ indicated at 301 and 302, respectively, indicate a change of almost 1% in FiO₂ (e.g., delivered from ventilator 210 as measured by an oxygen sensor of ventilator 210). The measurements indicated at 301a and 302a represent the change in FiO₂ that would be reflected in measurements in mixing chamber(s) 240, 250 between a first sampling time during interval A for inhalation mixing chamber 250 and when mixing chamber 240 started sampling exhalation gases during time interval B (for exhalation measurements). In an embodiment, the change in FiO₂ delivered, noted by “delta” near time interval B in FIG. 3, may be determined as the difference in FiO₂, e.g., a difference in the means of FiO₂ measurements or values for 301 and 302 during times A and B, respectively. Determination of this change can guide an embodiment in correcting measured oxygen gas concentration in mixing chamber(s) 240, 250, e.g., to correct the FiO₂ value of inspired oxygen, as indicated by application of the delta to measurement 301a to produce value 303, to help increase the reliability of VO₂ measurement.

By way of example, an embodiment may correct FiO₂ values to account for drift in FiO₂ delivered from ventilator 210 to increase reliability of VO₂ measurement, which is important given that inspired oxygen concentration varies in many systems delivering supplemental oxygen. A mixing chamber, e.g., 240, 250, allows for the computation of the volume of oxygen flowing through it by measuring oxygen gas concentrations (volume of oxygen in exhalation if chamber 240, exposed to exhalation gases, volume of oxygen gas inhaled if chamber 250, exposed to inhalation gases). VO₂ is computed as a difference of volume of inhaled and exhaled oxygen, as described in herein. However, mixing chamber(s) in a VO₂ monitoring device 270 is/are exposed to inhalation gases and exhalation gases at different times for each breathing cycle. FiO₂ variation (as emanating from ventilator 210) in conjunction with mixing chamber sampling of inhalation and exhalation gases at different times yields determinations of VO₂ not consistent with patient's actual oxygen consumption. An embodiment is designed to correct the inhalation oxygen concentration, measured in the example system of FIG. 2 by mixing chamber 250 containing an oxygen sensor 250a, in proportion to the change in FiO₂ delivered (A given FiO₂ drift), such that corrected FiO₂ for mixing chamber 250 reflects the actual FiO₂ at the time when mixing chamber 240 is sampling exhalation gases (time period B).

An example test bed with a VO₂ monitoring device, e.g., illustrated monitoring device 270, was used to validate using the correction (using Haldane transformation) to compute VO₂ as a difference of volume of oxygen inhaled less volume of oxygen exhaled, measured during time interval A and B, respectively, as shown in the Equation below:

$$V {O}_{2} = {\dot{V}}_{e} [ F i {O}_{2 MC}^{A} γ - F e {O}_{2 MC}^{B} ]$$

where:
\dot{V}ₑ: Exhale minute ventilation
FiO_{2MC}^{A}: inspired oxygen concentration measured during time interval A by mixing chamber
FeO_{2MC}^{B}: expired oxygen concentration measured during time interval B by mixing chamber

$$γ = \frac{1 - F e {O}_{2 MC}^{B} - F e C {O}_{2 MC}^{B}}{1 - F i {O}_{2 MC}^{A} - F i C {O}_{2 MC}^{A}}$$

As described herein, given FiO₂ supplementation, measured FeO₂ by mixing chamber 240 relates to a different value of FiO₂ and the computed VO₂ should be corrected for this difference, i.e., the change due to time variation or drift in FiO₂ produced by ventilator 210. A suitable correction includes subtracting the difference, e.g., detected at 103 of FIG. 1, from the measured FiO₂ at time period B at mixing chamber 240. Then, VO₂ may be computed using the corrected FiO₂ measurement by adjusting the measurement provided by mixing chamber 240. In an embodiment, VO₂ is therefore determined by:

$$V {O}_{2} = {\dot{V}}_{e} [ F i {O}_{2 MC}^{A} {γ}_{corrected} - F e {O}_{2 MC}^{B} - Δ \cdot {γ}_{corrected} ]$$

where:

$${γ}_{corrected} = \frac{1 - F e {O}_{2 MC}^{B} - F e C {O}_{2 MC}^{B}}{1 - F i {O}_{2 MC}^{A} + Δ - F i C {O}_{2 MC}^{A}}$$

FIG. 3 visually depicts this correction to FiO₂ to account for ventilator drift in systems that measure inhalation oxygen and exhalation oxygen at different times, e.g., using the same or different mixing chambers. The example of FIG. 3 provides simulated patient data and as shown, the change or drift in ventilator supplied FiO₂, noted by “delta” near time interval B in FIG. 3, is countered by a change, adjustment or correction to the FiO₂ value indicated at 301a. That is, the change or drift in supplied FiO₂ is detectable by measuring (point or average) FiO₂ at time A and time B, which indicates in this example a negative change or drop in FiO₂ provided by the ventilator between time A and time B. Thus, a correction of FiO₂ at time A, represented by the delta for FiO₂ delivered by the ventilator in FIG. 3, is for example applied to 301a, and is used to calculate VO₂. Thus, value 303 represents a substitute, synthetic or simulated value for measured O₂ in the mixing chamber(s), for example mixing chamber 240 at time period A, useful in correcting VO₂ systems (and respective oxygen sensor measurements) reliant on simultaneous or unchanging FiO₂ measurements, such as patient ventilation systems that need to be corrected due to supplying supplemental yet varying FiO₂. In this regard, it is noted that although FIG. 3 illustrates an example correction of an oxygen sensor measurement at time A, in an embodiment correction of one or more of oxygen sensor measurement values may be utilized. For example, in an embodiment, a correction to one or more of the FiO₂ value and the FeO₂ value may be determined based on a determined change in FiO₂. Further, an adjustment to one or more of the FiO₂ value and the FeO₂ value based on the correction(s) may be applied, so long as the change or drift in FiO₂ value is accounted for.

Table 1 illustrates the measured effectiveness of the proposed embodiments in VO₂ determination accuracy against ground truth measurement on a test bench composed of a mechanical ventilator and a test lung modified to accommodate for metabolic gases (O₂ and CO₂). In the bench, highly accurate mass flow controllers bleed a constant amount of O₂ and CO₂ into the test lung, which leads to realistic O₂ and CO₂ expiratory waveforms, with the caveat that O₂ is produced instead of being consumed. Although opposite in sign, the resulting VO₂ is a reliable ground truth against which devices and algorithms for its measurement can be tested and evaluated. By correcting for the change in measured oxygen given ventilator drift (Δ), VO₂ determined against ground truth VO₂ measurement is improved significantly. An embodiment therefore helps in correcting VO₂ where FiO₂ delivered is varying, resulting in robust VO₂ monitoring device performance independent of the need for stable (invariable) oxygen supply.

TABLE 1
Percent error for test bench

| | % VO₂ error |
|---|---|
| Method | (versus ground truth) |
| No Correction | −51.8 |
| With Correction | 12.5 |

Reduced error in VO₂ has many practical applications. For example, an embodiment may supply a more accurate measure of VO₂ that influences a decision support system. By way of example, should VO₂ measurement be used, for instance, to tailor nutritional therapy, a VO₂ reading with no correction may lead to a significant decrease (e.g., halving) in the amount of calories administered to the patient, compared to the patient's actual needs, leading to severe underfeeding. In an embodiment, a VO₂ measurement with correction would maintain the caloric target unaltered or slightly increased, without causing underfeeding.

An embodiment may include an application program configured to execute computer program instructions, for example as outlined in FIG. 1, which in combination with device hardware, permit identification or determination of correction(s) to measurement data of oxygen measurement sensors. Such an embodiment permits the ventilator system to update determinations of VO₂ and related outputs, such as indications, warnings, alerts, and communications to systems, healthcare providers or other users.

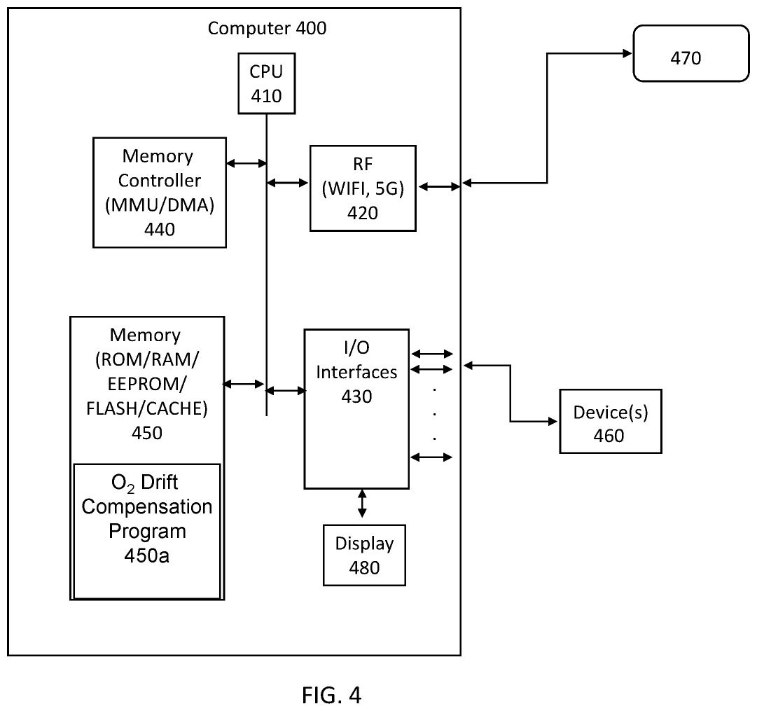

Referring to FIG. 4, it will be readily understood that certain embodiments can be implemented using any of a wide variety of devices or combinations of devices and components. In FIG. 4 an example of a computer 400 and its components are illustrated, which may be used in a device for implementing the functions or acts described herein, e.g., performing corrections to oxygen sensor measurements to account for ventilator drift in supplemental fractional inspired oxygen. Also, circuitry other than that illustrated in FIG. 4 may be utilized in one or more embodiments. The example of FIG. 4 includes certain functional blocks, as illustrated, which may be integrated onto a single semiconductor chip to meet specific application requirements.

One or more processing units are provided, which may include a central processing unit (CPU) 410, one or more graphics processing units (GPUs), and/or micro-processing units (MPUs), which include an arithmetic logic unit (ALU) that performs arithmetic and logic operations, instruction decoder that decodes instructions and provides information to a timing and control unit, as well as registers for temporary data storage. CPU 410 may comprise a single integrated circuit comprising several units, the design and arrangement of which vary according to the architecture chosen.

Computer 400 also includes a memory controller 440, e.g., comprising a direct memory access (DMA) controller to transfer data between memory 450 and hardware peripherals. Memory controller 440 includes a memory management unit (MMU) that functions to handle cache control, memory protection, and virtual memory. Computer 400 may include controllers for communication using various communication protocols (e.g., I²C, USB, etc.).

Memory 450 may include a variety of memory types, volatile and nonvolatile, e.g., read only memory (ROM), random access memory (RAM), electrically erasable programmable read only memory (EEPROM), Flash memory, and cache memory. Memory 450 may include embedded programs, code and downloaded software, e.g., an oxygen drift compensation program 450a that provides coded methods such as illustrated and described in connection with FIG. 1. By way of example, and not limitation, memory 450 may also include an operating system, application programs, other program modules, code, and program data, which may be downloaded, updated, or modified via remote devices.

A system bus permits communication between various components of the computer 400. I/O interfaces 430 and radio frequency (RF) devices 420, e.g., WIFI and telecommunication radios, may be included to permit computer 400 to send data to and receive data from remote devices using wireless mechanisms, noting that data exchange interfaces for wired data exchange may be utilized. Computer 400 may operate in a networked or distributed environment using logical connections to one or more other remote computers or devices 470, such as a ventilator, mixing chamber, oxygen sensor(s), or a combination of the foregoing. The logical connections may include a network, such local area network (LAN) or a wide area network (WAN) but may also include other networks/buses. For example, computer 400 may communicate data with and between peripheral device(s) 460, for example oxygen sensor(s).

Computer 400 may therefore execute program instructions or code configured to obtain, store, and analyze oxygen sensor data and perform other functionality of the embodiments, such as described in connection with FIG. 1. A user can interface with (for example, enter commands and information) the computer 400 through input devices, which may be connected to I/O interfaces 430. A display 480 or other type of output device may be connected to or integrated with the computer 400, for example via an interface selected from I/O interfaces 430.

It should be noted that the various functions described herein may be implemented using instructions or code stored on a memory, e.g., memory 450, that are transmitted to and executed by a processor, e.g., CPU 410. Computer 400 includes one or more storage devices that persistently store programs and other data. A storage device, as used herein, is a non-transitory computer readable storage medium. Some examples of a non-transitory storage device or computer readable storage medium include, but are not limited to, storage integral to computer 400, such as memory 450, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations according to various embodiments may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections or through a hard wire connection, such as over a USB connection.

One example embodiment therefore provides a ventilator system, comprising: a ventilator (210) comprising a first sensor, such as an oxygen sensor of ventilator. The system includes one or more mixing chambers (240, 240), with one or more other sensors disposed in the one or more mixing chambers, e.g., oxygen sensor(s) associated with the mixing chamber(s). The system includes a set of one or more processors (410); and a non-transitory storage medium (450) storing code (450a) executable by the set of one or more processors.

In an embodiment the code comprises code that obtains a first fractional inspired oxygen concentration (FiO₂) value of breathable gas provided by the ventilator to a patient, measured by the first sensor at a first time. In an embodiment, code obtains a second FiO₂ value of breathable gas provided by the ventilator to the patient, measured by the first sensor at a second time, and code obtains a third fractional FiO₂ value of breathable gas provided by the ventilator to the patient, measured by the one or more other sensors at the first time. In an embodiment the system obtains a fractional expired oxygen concentration (FeO₂) value of breathable gas exhaled by the patient, measured by the one or more other sensors at the second time.

In an embodiment, the system determines a change in FiO₂ between the first FiO₂ value and the second FiO₂ value, determines a correction to one or more of the third FiO₂ value and the FeO₂ value based on the determined change in FiO₂; and adjusts one or more of the third FiO₂ value and the FeO₂ value based on the correction. In an embodiment, the system determines a difference between: (a) the third FiO₂ value and an adjusted FeO₂ value; (b) an adjusted third FiO₂ value and the FeO₂ value; or (c) the adjusted third FiO₂ value and the adjusted FeO₂ value to determine VO_{2.}

In an embodiment, the first time, e.g., time A of FIG. 3, occurs during a first time period comprising inspiration by the patient and the second time, e.g., time B of FIG. 3, occurs during a second time period comprising expiration by the patient. The first time may include a first time sampling window, where the first and third FiO₂ values are average values over the first time sampling window. In an embodiment, the first time comprises a second time sampling window, where the second FiO₂ value and the FeO₂ value are average values over the second time sampling window.

In an embodiment, the first and second time sampling windows each comprise a plurality of breathing cycles by the patient, where the first and second times are separated by a plurality of breathing cycles, e.g., as indicated in FIG. 3.

In an embodiment, the system adjusts by outputting a correction value to increase or decrease the third FiO₂ value by a magnitude that is equivalent to the difference between the first FiO₂ value measured at the first time and the second FiO₂ value measured at the second time.

In an embodiment, the correction includes a correction identified as a real-time change in the first FiO₂ value provided by the first sensor over time. The real-time change value may be derived from a time series of oxygen measurements by the first sensor comprising oxygen measurements made in the first and second times. In an embodiment, a ventilator or component thereof may be characterized, for example periodically, to determine FiO₂ drift and associate a predetermined FiO₂ drift value with the ventilator or component thereof, used to correct or adjust values such as fractional inspired oxygen measurements, used in association with determining oxygen consumption, as described herein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. The word “about” or similar relative term as applied to numbers includes ordinary (conventional) rounding of the number with a fixed base such as 5 or 10.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A ventilator system, comprising:
a ventilator comprising a first sensor;
one or more mixing chambers;
one or more other sensors disposed in the one or more mixing chambers;
a set of one or more processors; and
a non-transitory storage medium storing code executable by the set of one or more processors, the code comprising:
code that obtains a first fractional inspired oxygen concentration (FiO₂) value of breathable gas provided by the ventilator to a patient, measured by the first sensor at a first time;
code that obtains a second FiO₂value of breathable gas provided by the ventilator to the patient, measured by the first sensor at a second time;
code that obtains a third fractional FiO₂value of breathable gas provided by the ventilator to the patient, measured by the one or more other sensors at the first time;
code that obtains a fractional expired oxygen concentration (FeO₂) value of breathable gas exhaled by the patient, measured by the one or more other sensors at the second time;
code that determines a change in FiO₂ between the first FiO₂ value and the second FiO₂ value;
code that determines a correction to one or more of the third FiO₂ value and the FeO₂ value based on the determined change in FiO₂; and
code that adjusts one or more of the third FiO₂ value and the FeO₂ value based on the correction.

2. The system of claim 1, wherein the code comprises:
code that determines a difference between: (a) the third FiO₂ value and an adjusted FeO₂ value; (b) an adjusted third FiO₂ value and the FeO₂ value; or (c) the adjusted third FiO₂ value and the adjusted FeO₂ value.

3. The system of claim 2, wherein the code comprises code that determines an oxygen consumption of the patient based on the difference.

4. The system of claim 1, wherein the first time occurs during a first time period comprising inspiration by the patient and the second time occurs during a second time period comprising expiration by the patient.

5. The system of claim 4, wherein the first time comprises a first time sampling window, wherein the first and third FiO₂ values are average values over the first time sampling window.

6. The system of claim 4, wherein the second time comprises a second time sampling window, wherein the second FiO₂ value and the FeO₂ value are average values over the second time sampling window.

7. The system of claim 1, wherein the code that adjusts comprises code that outputs a correction value to increase or decrease the third FiO₂ value by a magnitude that is equivalent to the difference between the first FiO₂ value measured at the first time and the second FiO₂ value measured at the second time.

8. The system of claim 1, wherein the code that determines a correction comprises code that identifies a real-time change in the first FiO₂ value provided by the first sensor over time.

9. The system of claim 8, wherein the real-time change value is derived from a time series of oxygen measurements by the first sensor comprising oxygen measurements made in the first and second times.

10. The system of claim 5, wherein the first and second time sampling windows each comprise a plurality of breathing cycles by the patient, and wherein the first and second times are separated by a plurality of breathing cycles.

11. A computer implemented method of using one or more processors that execute instructions to perform actions, comprising:
obtaining a first fractional inspired oxygen concentration (FiO₂) value of breathable gas provided by a ventilator to a patient, measured by a first sensor at a first time;
obtaining a second FiO₂ value of breathable gas provided by the ventilator to the patient, measured by the first sensor at a second time;
obtaining a third fractional FiO₂ value of breathable gas provided by the ventilator to the patient, measured by one or more other sensors at the first time;
obtaining a fractional expired oxygen concentration (FeO₂) value of breathable gas exhaled by the patient, measured by the one or more other sensors at the second time,
determining a change in FiO₂ between the first FiO₂ value and the second FiO₂ value,
determining, using the set of one or more processors, a correction to one or more of the third FiO₂ value and the FeO₂ value based on the determined change in FiO₂; and
adjusting one or more of the third FiO₂ value and the FeO₂ value based on the correction.

12. The method of claim 11, further comprising:
determining a difference between: (a) the third FiO₂ value and an adjusted FeO₂ value; (b) an adjusted third FiO₂ value and the FeO₂ value; or (c) the adjusted FiO₂ value and the adjusted FeO₂ value.

13. The method of claim 12, wherein an oxygen consumption of the patient is determined based on the difference.

14. The method of claim 1, wherein the first time occurs during a first time period comprising inspiration by the patient and the second time occurs during a second time period comprising expiration by the patient.

15. A computer program product, comprising:
a non-transitory storage medium storing code executable by the set of one or more processors, the code comprising:
code that obtains a first fractional inspired oxygen concentration (FiO₂) value of breathable gas provided by a ventilator to a patient, measured by a first sensor at a first time;
code that obtains a second FiO₂ value of breathable gas provided by the ventilator to the patient, measured by the first sensor at a second time;
code that obtains a third fractional FiO₂ value of breathable gas provided by the ventilator to the patient, measured by one or more other sensors at the first time;
code that obtains a fractional expired oxygen concentration (FeO₂) value of breathable gas exhaled by the patient, measured by the one or more other sensors at the second time,
code that determines a change in FiO₂ between the first FiO₂ value and the second FiO₂ value,
code that determines a correction to one or more of the third FiO₂ value and the FeO₂ value based on the determined change in FiO₂; and
code that adjusts one or more of the third FiO₂ value and the FeO₂ value based on the correction.
